(19) United States
(12) Patent Application Publication
(10) Pub. No.: US 2026/0157636 A1
(43) Pub. Date: Jun. 11, 2026

(54) SCANNING TIP FOR PHOTOACOUSTIC-ULTRASOUND MINI PROBE

(71) Applicant: UNIST(ULSAN NATIONAL INSTITUTE OF SCIENCE AND TECHNOLOGY), Ulsan (KR)

(72) Inventor: Joon-Mo Yang, Ulsan (KR)

(73) Assignee: UNIST(ULSAN NATIONAL INSTITUTE OF SCIENCE AND TECHNOLOGY), Ulsan (KR)

(21) Appl. No.: 19/182,210

(22) Filed: Apr. 17, 2025

Related U.S. Application Data
(63) Continuation of application No. PCT/KR2023/016017, filed on Oct. 17, 2023.

(30) Foreign Application Priority Data
Oct. 17, 2022 (KR) 10-2022-0133574
Oct. 13, 2023 (KR) 10-2023-0136980

(51) Int. Cl. A61B 5/00

(52) U.S. Cl. CPC A61B 5/0095

(57) ABSTRACT

According to the present disclosure, an original structure of optical and ultrasonic elements as well as a probe design which enables the acquisition of optical-resolution photoacoustic endoscopic images by the way that ultrasonic beams may be emitted overlappingly (that is, collinearly) with respect to a laser beam axis along which the laser beams are emitted, in a situation where one or two transducers with acoustic focusing capability are arranged symmetrically with respect to the laser beam axis, to be described later below, to have a synthetic acoustic focusing capability, within the limited endoscopic probe space described above, by combining only an optical fiber, a GRIN lens, and a prism, has been derived.

### CROSS REFERENCE TO RELATED APPLICATIONS

This application is a continuation application of International Patent Application No. PCT/KR 2023/016017, filed on Oct. 17, 2023, which claims priority to and the benefit of Korean Patent Application No. 10-2022-0133574, filed on Oct. 17, 2022 and Korean Patent Application No. 10-2023-0136980, filed on Oct. 13, 2023. The prior applications are incorporated herein by reference in their entirety.

### FIELD

The present disclosure relates to a scanning tip for a photoacoustic-ultrasonic mini-probe that may be implemented having a thin long probe shape like an ultrasonic endoscope currently used in clinical practice and may be used for a medical tomography endoscope device capable of being inserted into a subject to provide a tomographic image of an area inside the subject.

### BACKGROUND

The present disclosure relates to a so-called integrated photoacoustic and ultrasonic endoscopy (PAE-EUS) mini-probe technology that may simultaneously provide photoacoustic endoscopy (PAE) imaging information while maintaining a function of general endoscopic ultrasound (EUS).

Conventional photoacoustic-ultrasonic probes have a problem in that a photoacoustic axis does not overlap an ultrasonic axis or an overlapping range is very narrow.

### SUMMARY

According to the present disclosure, an original structure of optical and ultrasonic elements as well as a probe design which enables the acquisition of optical-resolution photoacoustic endoscopic images by the way that ultrasonic beams may be emitted overlappingly (that is, collinearly) with respect to a laser beam axis along which the laser beams are emitted, in a situation where one or two transducers with acoustic focusing capability are arranged symmetrically with respect to the laser beam axis, to be described later below, to have a synthetic acoustic focusing capability, within the limited endoscopic probe space described above, by combining only an optical fiber, a GRIN lens, and a prism, has been derived.

According to an aspect of the present disclosure, a scanning tip for a photoacoustic-ultrasonic mini-probe, comprising a transducer base having a through-hole and having a first inclined surface located on one side of the through-hole and a second inclined surface located on the other side of the through hole to be mutually symmetrical about a central axis of the through-hole, a first ultrasonic transducer arranged on the first inclined surface, a second ultrasonic transducer arranged on the second inclined surface, an optical fiber having an end arranged to a rear space of the transducer base, the rear space being opposite to a front space where the first ultrasonic transducer and the second ultrasonic transducer are arranged with respect to the transducer base, and a prism configured to reflect a laser beam emitted from the end of the optical fiber to pass through the through-hole, is provided.

The scanning tip for the photoacoustic-ultrasonic mini-probe may further include a GRIN lens interposed between the end of the optical fiber and the prism.

The scanning tip for the photoacoustic-ultrasonic mini-probe may further include a first micro-coaxial cable connected to the first ultrasonic transducer, and a second micro-coaxial cable connected to the second ultrasonic transducer, wherein the first micro-coaxial cable is arranged in the front space of the transducer base where the first ultrasonic transducer and the second ultrasonic transducer are arranged, and the second micro-coaxial cable is bent to pass the rear space of the transducer base.

The scanning tip for the photoacoustic-ultrasonic mini-probe may further include a GRIN lens interposed between the end of the optical fiber and the prism, and a GRIN lens housing configured to fix the GRIN lens, wherein the second micro-coaxial cable is arranged in a groove defined in the GRIN lens housing.

The scanning tip for the photoacoustic-ultrasonic mini-probe may further include an optical fiber housing configured to fix the optical fiber, wherein the second micro-coaxial cable is arranged in a groove defined in the optical fiber housing.

The scanning tip for the photoacoustic-ultrasonic mini-probe may further include a scanning tip casing configured to accommodate the transducer base, the first ultrasonic transducer, the second ultrasonic transducer, the optical fiber, and the prism inside the scanning tip casing, and has an opening region corresponding to the through-hole, the first ultrasonic transducer, and the second ultrasonic transducer.

The scanning tip casing may have a tube shape of a preset length, and have, on a side, the opening region corresponding to the through-hole, the first ultrasonic transducer, and the second ultrasonic transducer.

The scanning tip casing may have an opening portion of which an end is open.

The scanning tip for the photoacoustic-ultrasonic mini-probe may further include an epoxy portion configured to seal the end of the scanning tip casing and the opening portion.

The scanning tip casing may have an epoxy injection hole adjacent to another end.

The scanning tip for the photoacoustic-ultrasonic mini-probe may further include an epoxy configured to fill an inside of the scanning tip casing through the epoxy injection hole.

Other aspects, features and advantages other than those described above will become apparent from the following detailed description, claims and drawings for practicing the disclosure.

These and/or other aspects will become apparent and more readily appreciated from the following description of the embodiments, the claims, and the accompanying drawings.

### BRIEF DESCRIPTION OF THE DRAWINGS

The above and other aspects and features of certain embodiments of the disclosure will be more apparent from the following description taken in conjunction with the accompanying drawings, in which:

FIG. 1A is a schematic view illustrating a distal structure of a photoacoustic-ultrasonic mini-probe according to an embodiment;

FIG. 1B is a cross-sectional view of the probe of FIG. 1A taken along an x-z plane which includes a central axis of the probe;

FIG. 1C is a cross-sectional view taken along an x-y plane at a point A-A′ of the probe illustrated in FIG. 1B;

FIG. 2A to FIG. 2N are views illustrating a process of manufacturing a distal part of the photoacoustic-ultrasonic mini-probe presented in FIG. 1, according to an embodiment;

FIG. 3A is a view illustrating only a transducer base and two ultrasonic transducers among the components illustrated in FIG. 1B;

FIG. 3B is a side view of an ultrasonic transducer implemented to have an acoustic focusing capability, according to an embodiment;

FIG. 3C is a plan view of FIG. 3B taken from above;

FIG. 4 is a view illustrating a state of a photoacoustic-ultrasonic mini-probe implemented from a scanning tip to a base portion of a probe, according to an embodiment; and

FIG. 5 is a schematic view illustrating a method of arranging wires of an ultrasonic transducer, according to an embodiment.

### DETAILED DESCRIPTION

Detailed reference will now be made to the embodiments, examples of which are illustrated in the accompanying drawings, where like reference numerals indicate like elements throughout. It should be understood that the present embodiments may take various forms and are not limited to the descriptions provided herein. Accordingly, the embodiments are described below with reference to the figures to explain aspects of the present disclosure. As used herein, the term “and/or” includes any and all combinations of one or more of the listed items. Throughout the disclosure, the expression “at least one of a, b, or c” indicates only a, only b, only c, both a and b, both a and c, both b and c, all of a, b, and c, or variations thereof.

The disclosure permits various modifications and encompasses numerous embodiments. Certain embodiments are illustrated in the accompanying drawings and described in detail in this written description. The effects and features of the disclosure, as well as methods for achieving them, will be described in greater detail with reference to the accompanying drawings, which depict specific embodiments. This disclosure may, however, be embodied in many different forms and should not be construed as limited to the embodiments set forth herein.

One or more embodiments will be described below in more detail with reference to the accompanying drawings. Components that are identical or correspond to each other are assigned the same reference numerals across all figures, and redundant descriptions are omitted.

It will be understood that when a component, such as a layer, a film, a region, or a plate, is referred to as being “on” another component, the component can be directly on the other component or intervening components may be present thereon. Sizes of elements in the drawings may be exaggerated or reduced for convenience of description. In other words, since sizes and thicknesses of components in the drawings are arbitrarily illustrated for convenience of explanation, the following embodiments are not limited thereto.

In the following embodiments, the x-axis, the y-axis, and the z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In an embodiment below, terms such as “first” and “second” are used herein merely to describe a variety of elements, but the elements are not limited by the terms. Such terms are used for the purpose of distinguishing one element from another element.

In an embodiment below, it will be further understood that the terms “comprises” and/or “comprising” used herein specify the presence of stated features or components, but do not preclude the presence or addition of one or more other features or components.

“A and/or B” as used herein may include “A,” “B,” or “A and B.” In addition, “at least one of A and B” may include “A,” “B,” or “A and B.”

It will be understood that when a layer, region, or component is referred to as being “connected” to another layer, region, or component, it may be “directly connected” to the other layer, region, or component or may be “indirectly connected” to the other layer, region, or component with other layer, region, or component therebetween. For example, it will be understood that when a layer, region, or component is referred to as being “electrically connected” to another layer, region, or component, it may be “directly electrically connected” to the other layer, region, or component or may be “indirectly electrically connected” to other layer, region, or component with other layer, region, or component therebetween.

In the following embodiments, the singular expression includes the plural unless the context clearly indicates otherwise.

In the following embodiments, it will be further understood that the terms “includes”, “has”, “including” and/or “having” used herein specify the presence of stated features or components, but do not preclude the presence or addition of one or more other features or components.

The present disclosure may be modified in various ways and has various embodiments, and certain embodiments are illustrated in the drawings and described in detail. Effects and features of the present disclosure and a method for achieving the effects and features will become clear with reference to the embodiments described in detail below together with the drawings. However, the present disclosure is not limited to the embodiments disclosed below but may be implemented in various forms.

Hereinafter, embodiments of the present disclosure are described in detail with reference to the attached drawings, and when description is made with reference to the drawings, the same or corresponding components are assigned the same reference numerals and redundant descriptions thereof are omitted.

In the following embodiments, the terms first, second, and so on are not used in a limited sense but are used for the purpose of distinguishing one component from another component.

In the following embodiments, the singular expression includes the plural expression unless the context clearly indicates otherwise.

In the following embodiments, the terms “include” or “have” mean that a feature or component described in the specification exists, and do not preclude the possibility that one or more other features or components may be added.

In the embodiments below, when a component is said to be “connected” to another component, this includes not only being directly connected to the other component, but also being indirectly connected to the other component through another component.

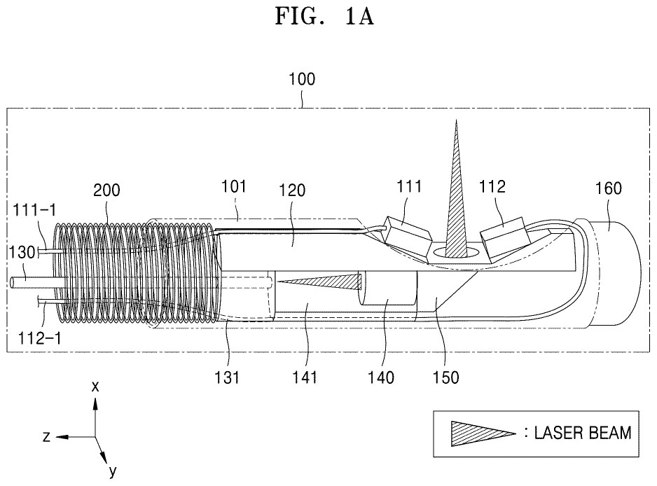

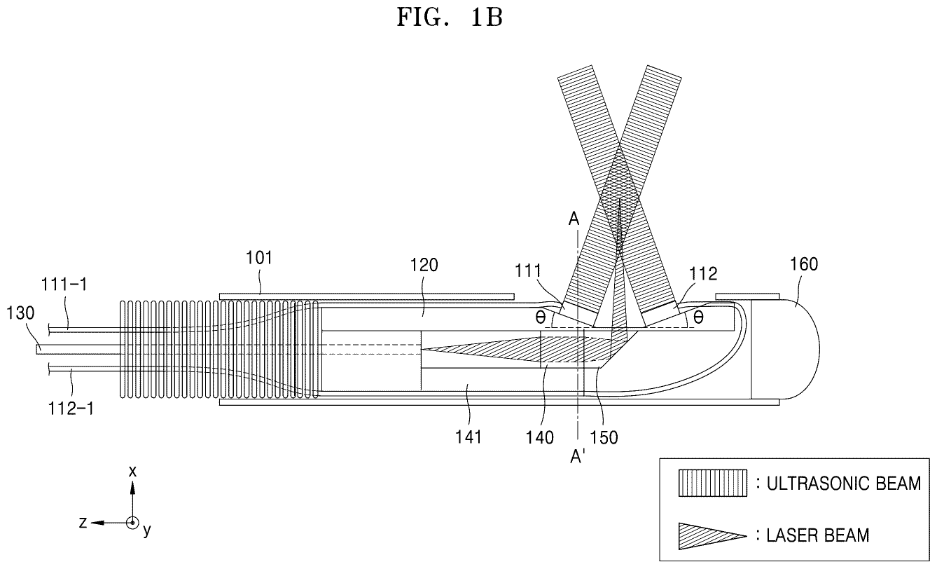

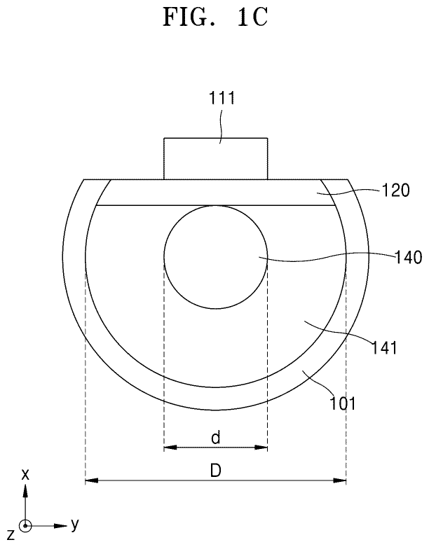

FIG. 1A is a schematic view illustrating a distal structure of a photoacoustic-ultrasonic mini-probe having both an optical-resolution photoacoustic imaging function and an ultrasonic imaging function while an optical axis is collinear with an acoustic axis, according to an embodiment, that is, a schematic view illustrating the entire configuration of a scanning tip 100 and the related operating concept. FIG. 1B is a cross-sectional view of the probe of FIG. 1A taken along an x-z plane which includes a central axis of the probe, and FIG. 1C is a cross-sectional view of the probe of FIG. 1B taken along an x-y plane at point A-A′ of the probe illustrated in FIG. 1B.

Referring to FIGS. 1A to 1C, the scanning tip 100 of the optical-resolution photoacoustic-ultrasonic mini-probe derived by the present disclosure includes a transducer base 120, a first ultrasonic transducer 111, a second ultrasonic transducer 112, an optical fiber 130, a GRIN (gradient-index) lens 140, and a prism 150. The first ultrasonic transducer 111 and the second ultrasonic transducer 112 are respectively arranged on two inclined surfaces formed to have a preset inclination angle θ with respect to an axis z of an endoscope probe. In this case, the first ultrasonic transducer 111 and the second ultrasonic transducer 112 may be arranged symmetrically with respect to a central axis of a through-hole of the transducer base 120 from which a laser beam is emitted. The optical fiber 130 may receive a laser pulse, which is emitted from a laser light source (not illustrated) provided separately in the outside, from a device to which the probe is connected, and guide the laser pulse to the scanning tip 100. The position of the optical fiber 130 may be fixed by the optical fiber housing 131. An end of the optical fiber 130 may be placed at a rear space of the transducer base 120 (i.e., in a direction to face away from the first ultrasonic transducer 111 and the second ultrasonic transducer 112 with respect to the transducer base 120). The rear space of the transducer base 120 may be opposite to a front space of the transducer base 120 where the first ultrasonic transducer 111 and the second ultrasonic transducer 112 are arranged. The GRIN lens 140 enables the laser beam emitted from the end of the optical fiber 130 to be focused on a preset working distance. The GRIN lens 140 may be fixed in position by the GRIN lens housing 141. The prism 150 may cause the laser beam, which is refracted in the form of being collected by the GRIN lens 140, to change its direction by 90° with respect to a central axis of the probe.

The components described above are surrounded by a scanning tip casing 101, and a torque coil 200 having a preset length is connected to an end of the scanning tip casing 101, and a first micro-coaxial cable 111-1 connected to the first ultrasonic transducer 111, a second micro-coaxial cable 112-1 connected to the second ultrasonic transducer 112, and the optical fiber 130 pass through the inside of the torque coil 200. In addition, an epoxy portion 160 is formed at the other end of the scanning tip casing 101 to prevent an acoustic matching fluid, such as water or oil, from penetrating into the inside of the scanning tip 100.

The optical fiber 130 located inside the torque coil 200 that is formed to range up to a proximal portion of the probe may guide the laser pulse received from a device connected to the probe to the scanning tip 100. When reaching the distal end of the optical fiber 130, the laser pulse is emitted at an angle determined according to an optical numerical aperture (NA) of the optical fiber 130. Thereafter, the laser pulse travels in an inner space of the GRIN lens housing 141 filled with air to an incident surface of the GRIN lens 140, is refracted by a lens effect within the GRIN lens 140, and finally travels inside the prism 150, changes its direction by 90° by a total reflection principle, and is focused into an acoustic matching fluid in which the probe is immersed. That is, because the laser beam is reflected from an inclined surface of the prism 150 by the total reflection principle, a space forming a boundary with the inclined surface of the prism 150 has to be filled with air. That is, an epoxy portion 160 serves to trap the air inside the scanning tip 100. However, when reflective coating is formed on the inclined surface of the prism 150, the total reflection principle does not need to be applied, and accordingly, it does not necessarily have to come into contact with air. For reference, the prism 150 illustrated in FIG. 1A and FIG. 1B has a part of one leg removed to provide a path for the second micro-coaxial cable 112-1 connected to the second ultrasonic transducer 112 to pass therethrough, but the present disclosure is not limited thereto.

When the scanning tip 100 illustrated in FIG. 1A and FIG. 1B is used, an optical focusing capability is provided to enable optical-resolution photoacoustic imaging to be performed, and because the first ultrasonic transducer 111 is symmetric to the second ultrasonic transducer 112, a certain level of acoustic focusing capability also can be achieved during an ultrasonic imaging process. Also, in order to maximize a signal-to-noise ratio in an optical-resolution photoacoustic imaging mode, it is preferable to form a focus of the laser beam at a point where the ultrasonic beam emitted by the first ultrasonic transducer 111 and the ultrasonic beam emitted by the second ultrasonic transducer 112 intersect each other. In this case, the working distance of an endoscopic probe can be adjusted by an inclination angle θ, by which the first ultrasonic transducer 111 and the second ultrasonic transducer 112 are attached obliquely, a pitch of the GRIN lens 140, and a distance between a tip of the optical fiber 130 and the incident surface of the GRIN lens 140, and may be optimized through appropriate adjustment thereof according to each application. Also, when such a high-resolution photoacoustic image at an optical-resolution level is not necessary, the GRIN lens 140 may be excluded.

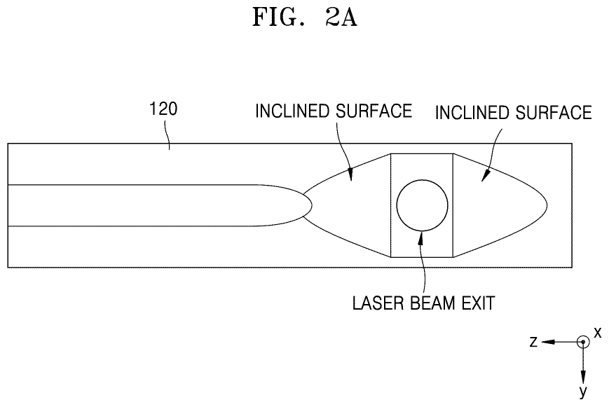

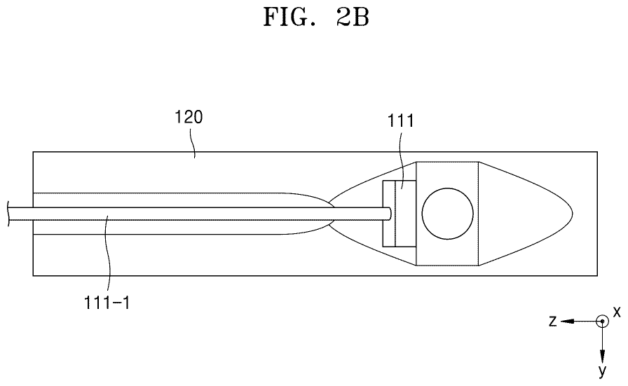

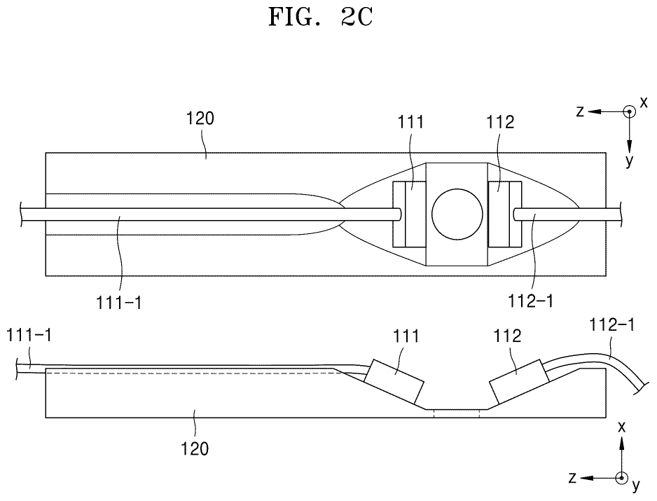

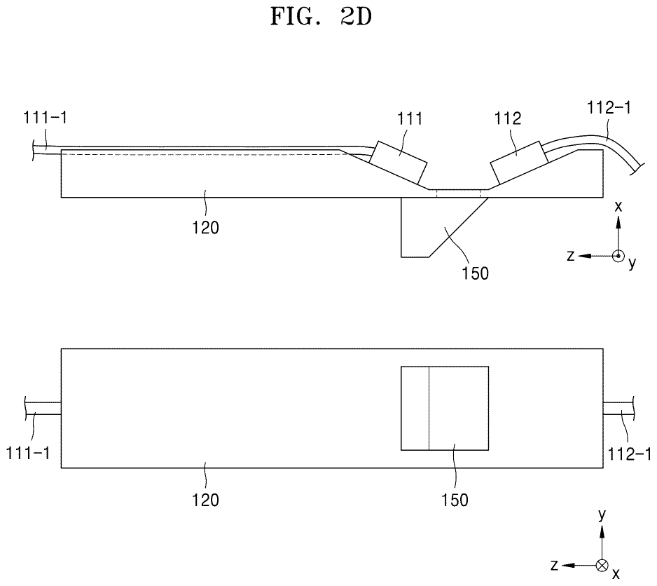

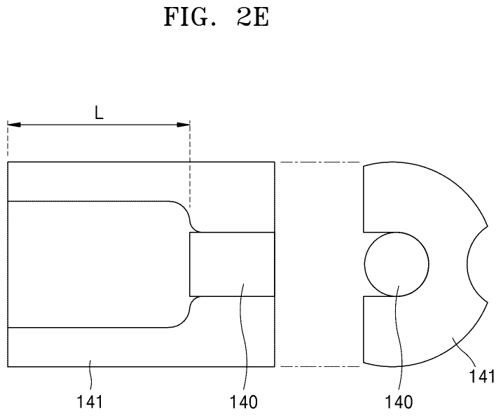

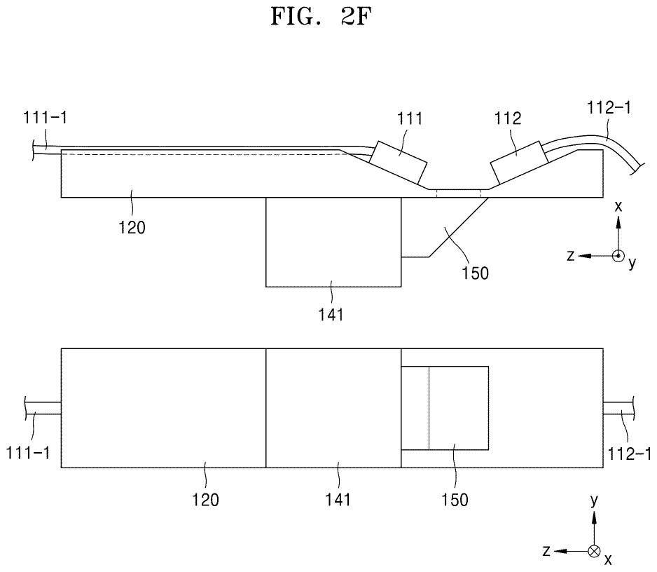

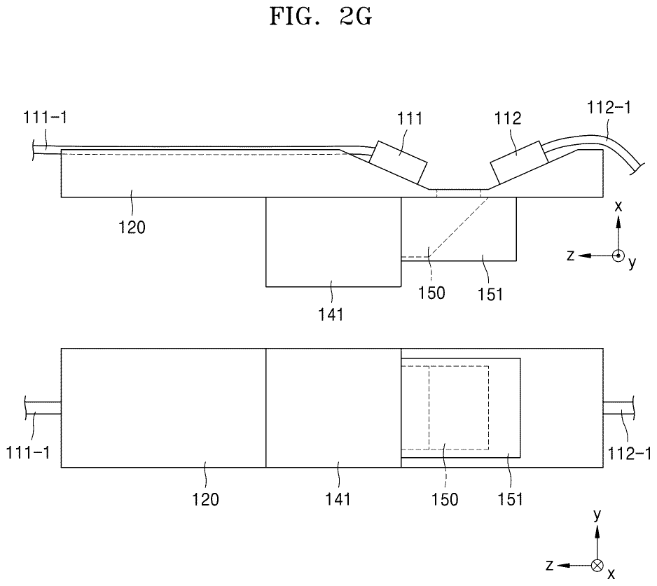

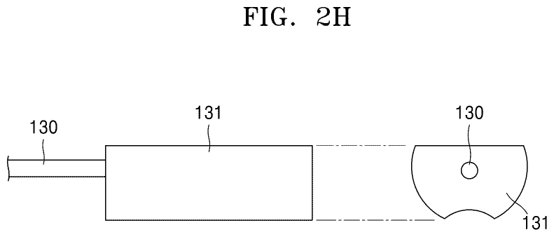

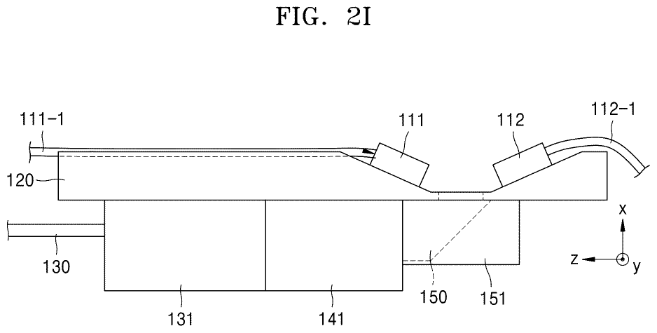

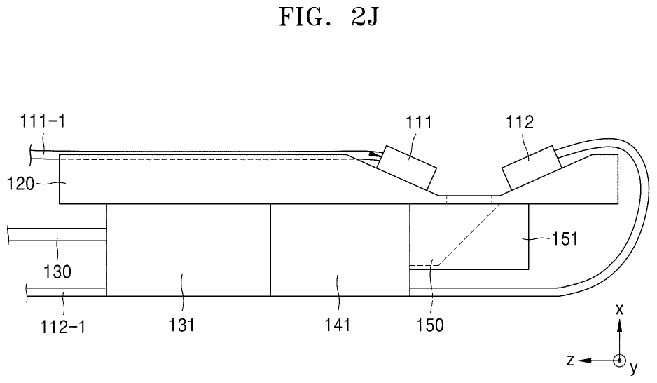

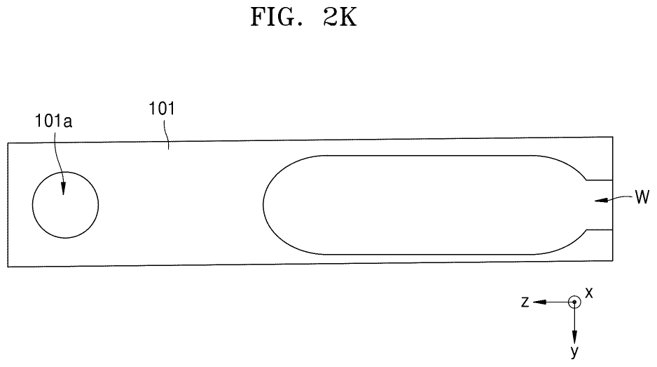

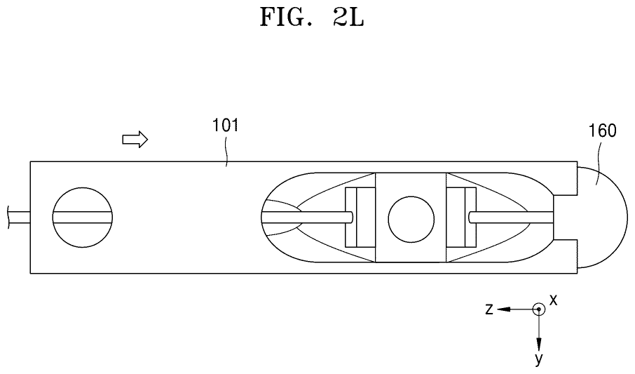

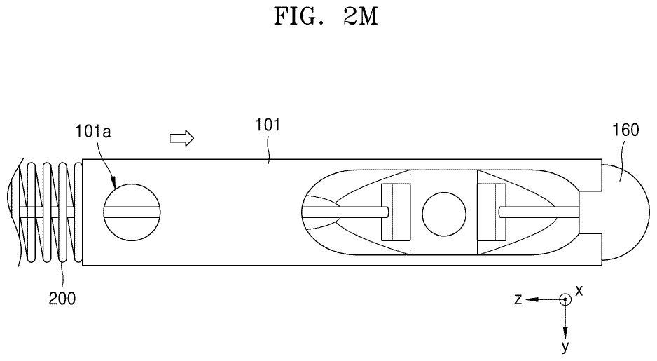

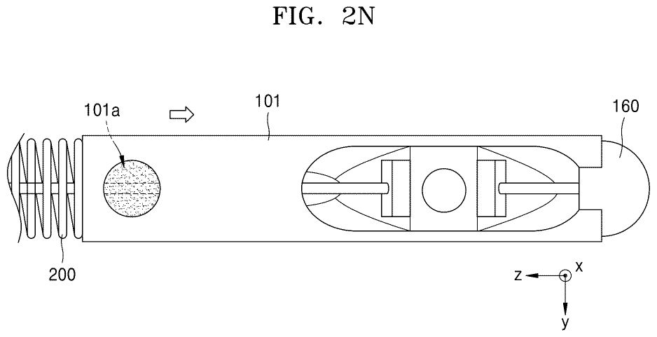

Hereinafter, a method of actually assembling and manufacturing the scanning tip 100 is described in 11 steps with reference to FIG. 2A to FIG. 2N.

As illustrated in FIG. 2A, which is a plan view, the transducer base 120 machined into a shape to have inclined surfaces symmetrical to each other with respect to an exit (that is, a through-hole) from which a laser beam is emitted according to the shape illustrated in FIG. 1A is prepared in step 1. Here, a diameter of the exit from which the laser beam is emitted has to be equal to or less than a width of the prism described below.

In step 2 illustrated in FIG. 2B which is a plan view, an adhesive is applied to one inclined surface to which the first ultrasonic transducer 111 and the first micro-coaxial cable 111-1 connected to each other is attached. For this, the first micro-coaxial cable 111-1 may be arranged in the front space of the transducer base 120 where the first ultrasonic transducer 111 and the second ultrasonic transducer 112 are arranged.

An assembly process is described in detail with reference to FIG. 2C to FIG. 2H which illustrate views taken from two different directions.

In step 3 illustrated in FIG. 2C illustrating a plan view and a side view, an adhesive is applied to a second inclined surface, which is opposite to a first inclined surface to which the first ultrasonic transducer 111 is attached, and the second ultrasonic transducer 112 and the second micro-coaxial cable 112-1 connected to each other are attached to the second inclined surface.

In step 4 illustrated in FIG. 2D illustrating a side view and a bottom view, an adhesive is applied to a rear surface (a bottom surface or an opposite surface) of the transducer base 120 to which the first ultrasonic transducer 111 and the second ultrasonic transducer 112 are attached, and the prism 150 is attached the rear surface. In this case, a position of the prism 150 has to be accurately determined in such a way that one of two vertical surfaces of the prism 150 includes a laser beam exit (that is, a through-hole), and in addition, a circular edge of the laser beam exit has to be sealed with an adhesive so that an acoustic matching fluid does not permeate into an optical system inside the scanning tip 100.

Meanwhile, as illustrated in FIG. 2E illustrating a plan view and a side view, a GRIN lens module composed of the GRIN lens 140 and the GRIN lens housing 141 pre-prepared in a separate step is placed in close contact with the other surface of the two vertical surfaces of the prism 150 according to step 5 illustrated in FIG. 2F, which is a side view and a bottom view. In this case, it is preferable to apply an adhesive to a flat surface on which the GRIN lens housing 141 is coupled to the transducer base 120 rather than to a surface on which the GRIN lens 140 is in contact with the prism 150.

As illustrated in the plan view on the left side of FIG. 2E, a length of the GRIN lens housing 141 of the GRIN lens module may be formed to be much longer than a length of the GRIN lens 140 by a length L, which is an interval to set a desired working distance by appropriately controlling the length parameter L. A parameter D (see FIG. 1C) of the GRIN lens module illustrated in the right side view on the right side of FIG. 2E represents a diameter of the entire GRIN lens module, and a parameter d (see FIG. 1C) represents a diameter of the GRIN lens 140 only. As illustrated in FIG. 2E, the GRIN lens housing 141 may partially surround the GRIN lens 140 without completely surrounding the GRIN lens 140. This may be understood as one side of the GRIN lens module is removed by a thickness of (D-d)/2. By doing in this way, a portion in which the GRIN lens module is partially removed may be used to effectively affix the GRIN lens module onto the flat rear surface of the transducer base 120 so that the position of the GRIN lens 140 can be precisely placed on a central axis of the scanning tip 100.

In step 6 illustrated in FIG. 2G illustrating a side view and a bottom view, the prism 150 is covered by a prism cover 151 having a cross-section in the shape of a Korean letter “⊏” to prevent foreign substances from adhering to an inclined surface of the prism 150 during a subsequent assembly process. The step 6 is not essential, and accordingly, the step 6 may be omitted.

Meanwhile, as illustrated in FIG. 2H illustrating a side view and a front view, an optical fiber module composed of an optical fiber 130 and the optical fiber housing 131 pre-prepared in a separate step is attached to the remaining flat surface of the transducer base 120 so as to be in close contact with the GRIN lens module in step 7 illustrated in FIG. 2I illustrating a side view. The optical fiber housing 131 of the optical fiber module may also be partially removed by a thickness of (D-d)/2, as in the case of the GRIN lens module described above. This is to ensure that a central axis of the optical fiber 130 is aligned with a central axis of the GRIN lens 140. In a situation where a value of the length L described above is determined in advance, the end of the optical fiber 130 has to be placed exactly on an end surface of the optical fiber housing 131 (that is, a surface indicated in the front view of FIG. 2H).

In step 8 illustrated in FIG. 2J which is a side view, the second micro-coaxial cable 112-1 is bent to pass to face away from the first ultrasonic transducer 111 and the second ultrasonic transducer 112 with respect to the transducer base 120 (i.e., through the rear space of the transducer base 120), and is arranged and fixed along grooves pre-formed (or pre-defined) along one surface of the GRIN lens housing 141 and one surface of the optical fiber housing 131. As explained, the grooves may be pre-formed (or pre-defined) on the surfaces of the GRIN lens housing 141 and the optical fiber housing 131 such that a wire may pass through the grooves to be buried therein, but the present disclosure is not limited thereto, and the grooves may not be formed.

Thereafter, as illustrated in a plan view of FIG. 2K, the scanning tip casing 101 of a tube shape having a preset length and an open region on a part of a side surface is prepared in advance in a separate step, and in step 9 illustrated in FIG. 2L, the components assembled in the previous step are moved in the direction of an arrow and the components assembled in the previous step are inserted into the scanning tip casing 101 and fixed by an adhesive. The scanning tip casing 101 has an open region on a side surface, and a through-hole of the transducer base 120, the first ultrasonic transducer 111, and the second ultrasonic transducer 112 are arranged to correspond to the open region. Of course, when inserting the components assembled in the previous step into the scanning tip casing 101, the optical fiber 130 having a preset length, the first micro-coaxial cable 111-1, and the second micro-coaxial cable 112-1 have to be passed first into the scanning tip casing 101.

The scanning tip casing 101 may have an open portion W formed in an open shape at the end, and the opening portion W is formed in advance to allow the second ultrasonic transducer 112 to pass therethrough smoothly when the components assembled in the previous step are inserted into the opening portion W. Also, in some cases, for example, when an inner diameter of the scanning tip casing 101 is sufficiently large, the shaping process of the relevant opening portion W may be omitted.

When the components assembled in the previous step are inserted into the scanning tip casing 101 and fixed by an adhesive, the adhesive serves not only to fix the components but also to prevent an acoustic matching fluid in which a probe will be immersed in the future from flowing into the probe, and accordingly, the adhesive is filled in all gaps to seal all the gaps firmly. The epoxy portion 160 performs the sealing role at an end point of the probe. The epoxy portion 160 may seal not only the end of the scanning tip casing 101 but also the entire opening portion W. For reference, the scanning tip casing 101 could have an epoxy injection hole 101a, which will be explained later.

In step 10 illustrated in a plan view of FIG. 2M, the torque coil 200 is inserted into the scanning tip casing 101 and fitted into the scanning tip casing 101. Of course, an appropriate amount of epoxy may be applied to an end of the torque coil 200 for adhesion to the scanning tip casing 101 just before the fitting process.

In step 11 illustrated in a plan view of FIG. 2N, by additionally applying epoxy through the epoxy injection hole 101a, which is pre-formed near the other end of the scanning tip casing 101, the epoxy can be injected into at least part of a space in the scanning tip casing (101), and accordingly, an acoustic matching fluid, in which the probe will be immersed in the future, is prevented from flowing into an inner space of the scanning tip 100 through an internal channel of the torque coil 200. Also, the epoxy injection hole may be omitted.

The method of manufacturing a distal structure of a photoacoustic-ultrasonic mini-probe according to an embodiment of the present disclosure is described above. Because the method described above is only one embodiment, the described order may be partially changed, and in some cases, some processes may be omitted or other processes may be added.

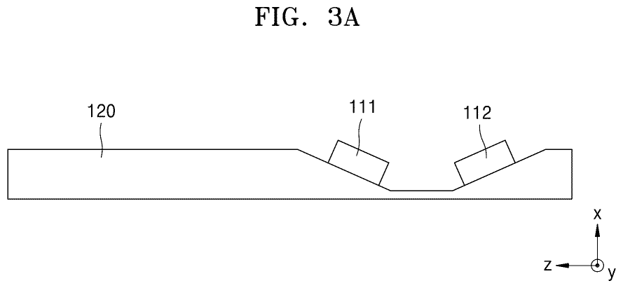

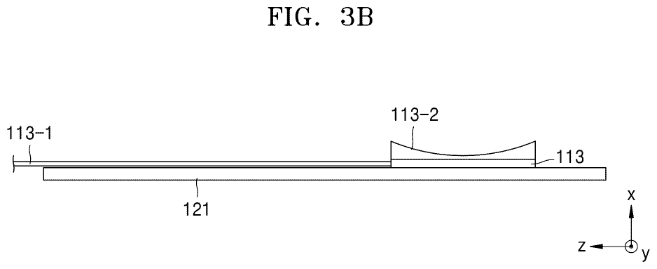

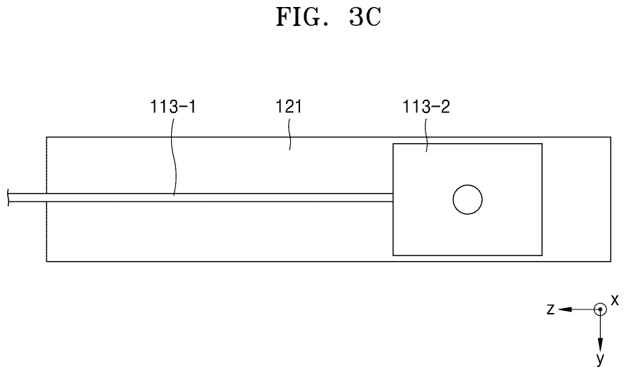

As described above, although the scanning tip with a synthetic acoustic focusing capability in a direction that is collinear with an axis from which a laser beam is emitted by using two ultrasonic transducers is presented according to one embodiment, the scanning tip of the photoacoustic-ultrasonic mini-probe according to the present disclosure may also be implemented based on a single focused ultrasonic transducer. This is described below with reference to FIG. 3A to FIG. 3C.

First, FIG. 3A is a view illustrating only the transducer base 120, the first ultrasonic transducer 111, and the second ultrasonic transducer 112 among the components illustrated in FIG. 1B, and FIG. 3B and FIG. 3C are views schematically illustrating a case where an ultrasonic transducer having a single piezoelectric element with an acoustic focusing capability is provided, unlike FIG. 3A. It may be understood that FIG. 3B is a side view and FIG. 3C is a plan view. As illustrated in FIG. 3B and FIG. 3C, a planar piezoelectric element 113 is provided on a planar transducer base 121 having a flat shape, and an acoustic lens 113-2 is bonded thereon to implement a scanning tip with an acoustic focusing capability. In this case, an electric signal input to and output from the planar piezoelectric element 113 is transmitted through a planar piezoelectric element cable 113-1, and a hole located at the center of the acoustic lens 113-2 illustrated in FIG. 3C is an exit through which a laser beam is emitted. An embodiment based on the single piezoelectric element may also be combined with other optical elements as illustrated in FIG. 1.

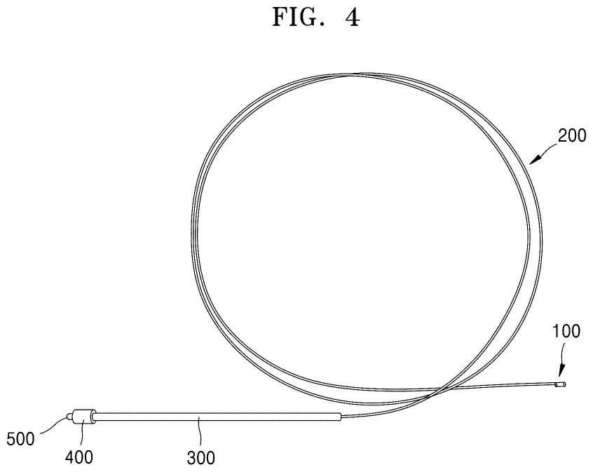

FIG. 4 is a view illustrating a state of a photoacoustic-ultrasonic mini-probe implemented from a scanning tip to a proximal portion of the probe, according to an embodiment.

The present disclosure is primarily intended to present a structure of a scanning tip capable of being used for a photoacoustic-ultrasonic mini-probe that may solve the problem described above, but various embodiments of the scanning tip presented above may additionally include several additional components illustrated in FIG. 4. FIG. 4 illustrates a torque coil 200 described above terminated in a form that includes a shaft 300 having a preset length to suit an application, an FC/PC connector 400, and a ceramic ferrule 500. In some cases, a length of the shaft 300 may determine a scan length of a three-dimensional pullback scan, and in another case, a ball bearing module may be added therearound. In addition, in another embodiment, the FC/PC connector 400 may be omitted and only the ceramic ferrule 500 may be included. In an embodiment that includes both the FC/PC connector 400 and the ceramic ferrule 500, an electrical path may also be added simultaneously to both components through a method such as conductive plating.

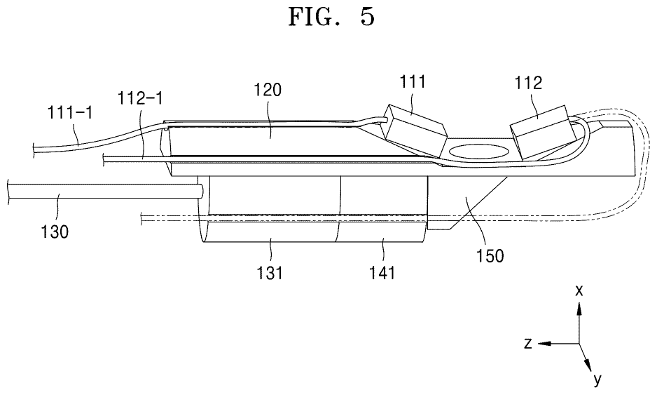

FIG. 5 is a schematic diagram illustrating a method of arranging wires of an ultrasonic transducer differently, according to an embodiment.

In FIG. 1A, FIG. 1B, FIG. 2J, FIG. 2L, FIG. 2M and FIG. 2N, the second micro-coaxial cable 112-1 connected to the second ultrasonic transducer 112 is illustrated as being arranged to pass along an outer center of the GRIN lens housing 141 and the optical fiber housing 131. However, when there is sufficient space, the second micro-coaxial cable 112-1 connected to the second ultrasonic transducer 112 may also pass through one side of the transducer base 120 as illustrated in FIG. 5. In addition, the second micro-coaxial cable 112-1 connected to the second ultrasonic transducer 112 may also pass along a path illustrated by two point-chain lines in FIG. 5, that is, through one side (+y direction) of the GRIN lens housing 141 and the optical fiber housing 131.

Because of dimensional restrictions (diameter: 0.7-1.5 mm, rigid distal length: less than 10 mm) that are commonly required in the relevant application fields of photoacoustic-ultrasonic mini-probes or catheters, it is important to effectively arrange optical and ultrasonic elements, which have to be provided, inside a distal end thereof, so-called the scanning tip. When a laser beam (that is, an optical axis) emitted from the distal end toward a lateral direction is misaligned with an acoustic axis formed by an ultrasound beam, and as a result, when tissues to be examined are not located at a distance where a laser beam axis intersects the acoustic axis, the detected signal is significantly reduced.

Also, when the related sound waves are transmitted via one or more reflective surfaces in such a configuration where an ultrasonic transducer is placed deep inside a probe rather than on a probe surface, and, serious distortion occurs in the sound waves during the sound waves propagate through a narrow space, and other problems, such as bubbles forming in a space inside the probe, may easily occur because the ultrasonic transducer is placed too deep inside the probe. In addition, it is not easy to prevent related optical components from being damaged despite the pulse laser with high instantaneous peak power in a limited space and to have an optical-resolution photoacoustic imaging capability that requires high-level optical focusing.

Of course, it would be also conceptually possible to reduce the size of any endoscopic probe with any type of structure, but in reality, it is never easy to actually implement the endoscopic probe within a limited dimension. For example, even when only the problem (that is, a process of arrangement) of designating a path for a wire connected to an ultrasonic transducer is considered, the related task may seem easy in concept, but the actual implementation is never simple. Because the ultrasonic transducer is approximately 1 mm or less in diameter and 0.3 mm or less in thickness, in a state where the micro-coaxial cable attached to the transducer for inputting and outputting electrical signals is approximately 150 μm or more in thickness, while the dimension of the entire endoscope probe is 0.7-1.5 mm in diameter, it is never a trivial matter to allocate a space for a cable having a thickness of 150 μm.

However, according to the unique structure and manufacturing method proposed by the present disclosure as described above, it is possible to simultaneously realize optical-resolution photoacoustic images and traditional ultrasonic images in a space of 0.7 to 1.5 mm in diameter and 10 mm or less in length of the distal scanning tip, while an optical axis is perfectly collinear with an acoustic axis, without any damage to the optical elements caused by the pulsed laser beam passing through a relevant region. In addition, because the ultrasonic transducer is placed on a surface of the probe, the ultrasonic transducer comes into more direct contact with the surrounding acoustic matching fluid, and accordingly, the probability that bubbles and so on adhere to the surface of the ultrasonic transducer during actual use to interfere with the related imaging procedure is significantly reduced.

According to the unique structure and manufacturing method proposed by the present disclosure as described above, it is possible to simultaneously obtain optical-resolution photoacoustic images and traditional ultrasonic images in a space of 0.7 to 1.5 mm in diameter and 10 mm or less in length of a rigid distal scanning tip, while an optical axis is perfectly collinear with an acoustic axis, without any damage to optical elements that may be caused by a pulsed laser beam passing through a relevant region. In addition, as an ultrasonic transducer is placed on a surface of a probe, the ultrasonic transducer comes into more direct contact with a surrounding acoustic matching fluid, and accordingly, a probability that bubbles and so on adhere to the surface of the ultrasonic transducer during actual use, which may interfere with the related imaging procedure, is significantly reduced.

The unique probe structure proposed by the present disclosure is mainly targeted at endoscopes for diagnosing digestive diseases but may be applied to cardiovascular disease diagnosis applications that require much higher probe miniaturization, as well as various other endoscope fields.

However, the scope of the present disclosure is not limited by these effects.

It should be understood that embodiments described herein should be considered in a descriptive sense and not for purposes of limitation. Descriptions of features or aspects within each embodiment should be considered as available for other similar features or aspects in other embodiments. While one or more embodiments have been described with reference to the figures, it will be understood by those of ordinary skill in the art that various changes in form and details may be made therein without departing from the spirit and scope as set forth by the following claims.

1. A scanning tip for a photoacoustic-ultrasonic mini-probe, the scanning tip comprising:
a transducer base having a through-hole and having a first inclined surface located on one side of the through-hole and a second inclined surface located on the other side of the through hole to be mutually symmetrical about a central axis of the through-hole;
a first ultrasonic transducer arranged on the first inclined surface;
a second ultrasonic transducer arranged on the second inclined surface;
an optical fiber having an end arranged to a rear space of the transducer base, the rear space being opposite to a front space where the first ultrasonic transducer and the second ultrasonic transducer are arranged with respect to the transducer base; and
a prism configured to reflect a laser beam emitted from the end of the optical fiber to pass through the through-hole.

2. The scanning tip for the photoacoustic-ultrasonic mini-probe of claim 1, further comprising a GRIN lens interposed between the end of the optical fiber and the prism.

3. The scanning tip for the photoacoustic-ultrasonic mini-probe of claim 1, further comprising:
a first micro-coaxial cable connected to the first ultrasonic transducer; and
a second micro-coaxial cable connected to the second ultrasonic transducer;
wherein the first micro-coaxial cable is arranged in the front space of the transducer base where the first ultrasonic transducer and the second ultrasonic transducer are arranged, and the second micro-coaxial cable is bent to pass the rear space of the transducer base.

4. The scanning tip for the photoacoustic-ultrasonic mini-probe of claim 3, further comprising:
a GRIN lens interposed between the end of the optical fiber and the prism; and
a GRIN lens housing configured to fix the GRIN lens;
wherein the second micro-coaxial cable is arranged in a groove defined in the GRIN lens housing.

5. The scanning tip for the photoacoustic-ultrasonic mini-probe of claim 3, further comprising:
an optical fiber housing configured to fix the optical fiber,
wherein the second micro-coaxial cable is arranged in a groove defined in the optical fiber housing.

6. The scanning tip for the photoacoustic-ultrasonic mini-probe of claim 1, further comprising a scanning tip casing configured to accommodate the transducer base, the first ultrasonic transducer, the second ultrasonic transducer, the optical fiber, and the prism inside the scanning tip casing, and has an opening region corresponding to the through-hole, the first ultrasonic transducer, and the second ultrasonic transducer.

7. The scanning tip for the photoacoustic-ultrasonic mini-probe of claim 6, wherein the scanning tip casing has a tube shape of a preset length, and has, on a side, the opening region corresponding to the through-hole, the first ultrasonic transducer, and the second ultrasonic transducer.

8. The scanning tip for the photoacoustic-ultrasonic mini-probe of claim 7, wherein the scanning tip casing has an opening portion of which an end is open.

9. The scanning tip for the photoacoustic-ultrasonic mini-probe of claim 8, further comprising an epoxy portion configured to seal the end of the scanning tip casing and the opening portion.

10. The scanning tip for the photoacoustic-ultrasonic mini-probe of claim 8, wherein the scanning tip casing has an epoxy injection hole adjacent to another end.

11. The scanning tip for the photoacoustic-ultrasonic mini-probe of claim 10, further comprising an epoxy configured to fill an inside of the scanning tip casing through the epoxy injection hole.
